(19) United States
(12) Patent Application Publication
(10) Pub. No.: US 2002/0022242 A1
(43) Pub. Date: Feb. 21, 2002

(54) Diagnostic test for alzheimer's disease

(76) Inventors: David Henry Small, Ashburton (AU); Javier Saez-Valero, Alicante (ES)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 09/829,446

(22) Filed: Apr. 9, 2001

Related U.S. Application Data
(60) Provisional application No. 60/195,231, filed on Apr. 7, 2000.

(51) Int. Cl. C12Q 1/66

(52) U.S. Cl. 435/8

(57) ABSTRACT

A method for the diagnosis of Alzheimer's disease (AD) in a patient, comprising the steps of:

(1) providing a sample of an appropriate body fluid from said patient, and

(2) detecting the presence of BuChE with an altered glycosylation pattern in said sample.

### CROSS-REFERENCE TO RELATED APPLICATIONS

The present application is based upon and claims the priority of U.S. Provisional Patent Application Ser. No. 60/195,231 filed Apr. 7, 2000 which is incorporated herein by reference.

### TECHNICAL FIELD

The present invention is concerned with a diagnostic test for Alzheimer's disease.

### BACKGROUND ART

Alzheimer's disease (AD) is a common progressive dementia involving loss of memory and higher cognitive function. The disease is characterized by the presence of amyloid deposits in the brains of sufferers. These deposits are found both extracellularly (amyloid plaques) and intracellularly (neurofibrillary tangles). The principal constituent of amyloid plaques is the amyloid protein (Aβ) which is produced by proteolytic cleavage for the amyloid protein precursor (APP) (Evin et al., 1994). The principal constituent of neurofibrillary tangles is the cytoskeletal protein tau (Kosik, 1992).

One of the characteristic neurochemical changes observed in AD is the loss of acetylcholinesterase (AChE) and choline acetyltransferase activity in regions of the brain such as the cortex, hippocampus, amygdala and nucleus basalis (Whitehouse et al., 1981, 1982; Struble et al., 1982; Mesulam and Geula, 1988). The loss of cholinergic structure and markers correlates with the number of plaque and tangle lesions present, as well as with the clinical severity of the disease (Perry et al., 1978; Wilcock et al., 1982; Neary et al., 1986; Perry, 1986). This loss of AChE is accompanied by an increase in butyrylcholinesterase (BuChE) (Atack et al., 1986).

Accurate diagnosis of AD during life is essential. However, clinical evaluation is at best only about 80% accurate. Therefore, there is a need to identify specific biochemical markers of AD. So far, analysis of blood or cerebrospinal fluid (CSF) has not yielded a biochemical marker of sufficient diagnostic value (Blass et al., 1998), although detectable differences are reported in the levels of certain proteins (Motter et al., 1995).

The assay of levels of AChE and BuChE activity in the blood and the cerebrospinal fluid (CSF) have been proposed as an ante mortem diagnostic test for AD. However, no consensus has been reached as to whether the levels of AChE and BuChE are consistently affected in these tissues. The level of serum or plasma AChE has been reported to be increased (Perry et al., 1982; Atack et al., 1985), decreased (Nakano et al., 1986; Yamamoto et al., 1990) or unchanged (St. Clair et al., 1986; Sirvio et al., 1989) in AD patients. The level of erythrocyte AChE has been reported as either unaffected (Atack et al., 1985; Perry et al., 1982) or decreased Chipperfield et al., 1981). The level of AChE activity in the CSF of AD patients has been reported to be decreased (most recently by Appleyard and McDonald, 1992; Shen et al., 1993) or unchanged (most recently by Appleyard et al., 1987; Ruberg et al., 1987).

AChE and BuChE have been shown to exist as up to six different molecular isoforms, three of which are the monomeric (G1), dimeric (G2) and tetrameric (G4) isoforms (Massoulié et al., 1993). The relative proportion of the different isoforms of AChE and BuChE are markedly affected in AD, with a decrease in the G4 isoform of AChE in the parietal cortex (Atack et al., 1983), and an increase in the G1 isoform of AChE (Arendt et al., 1992). Similar changes have been identified in other AD brain regions including Brodman areas 9, 10, 11, 21 and 40, as well as the amygdala (Fishman et al., 1986). Asymmetric collagen-tailed isoforms (A12) are increased by up to 400% in Brodman area 21, although they represent only a trace amount of the total AChE in the human brain (Younkin et al., 1986).

However, to date changes in AChE and BuChE expression and isoform distribution have not been found to be of sufficient sensitivity or specificity to be useful diagnostic markers of AD.

### DISCLOSURE OF THE INVENTION

There remains a need for a diagnostic test for AD based on a biochemical analysis of body fluids such as blood or CSF. The present invention provides such a test on the basis that the butyrylcholinesterase (BuChE) of AD patients shows a different glycosylation pattern to the BuChE of non-AD groups.

According to a first aspect of the present invention there is provided a method for the diagnosis of Alzheimer's disease (AD) in a patient, comprising the steps of:

(1) providing a sample of an appropriate body fluid from said patient, and

(2) detecting the presence of BuChE with an altered glycosylation pattern in said sample.

In one embodiment of the invention the relative proportion of BuChE with a specific glycosylation pattern to the total BuChE is measured.

Measurement of the relative proportion of the isoforms of BuChE with a specific glycosylation pattern to the total BuChE may be carried out in any convenient manner, for example, by using biochemical analysis techniques such as HPLC and mass spectrometry, or immunological techniques such as ELISA or, assays. However, a particularly preferred means of measuring the relative proportions of the isoforms of BuChE involves a lectin-binding analysis.

It has been established that on average approximately 93.6% of the BuChE in the CSF of AD patients binds to Concanavalin (Con A). Accordingly, in a particularly preferred embodiment of the invention, in order to detect the presence of BuChE with a to specific glycosylation pattern in the sample, the binding of BuChE to Con A is determined. The percentage of BuChE bound to Con A is characteristic of the proportion of BuChE with the specific glycosylation pattern.

Also, it is particularly useful to measure the activity of unbound BuChE in each experiment, by determining the amount of BuChE unbound to Con A relative to the total BuChE in the sample.

In another embodiment of the present invention, it is particularly advantageous to compare the ratio of AChE that binds to Con A with the AChE that binds to wheat germ agglutinin (WGA), hereinafter referred to as the C/W ratio, versus the percentage of BuChE unbound to Con A. The ratio is characteristic of the different glycosylation patterns of AChE. By plotting the C/W ratio versus the percentage BuChE unbound to Con A, the separation of patients diagnosed with AD as compared with non-AD becomes evident when viewing such a plot.

Approximately 75-95% of the AChE in the CSF of AD patients bind to Concanavalin (Con A) or wheat germ agglutinin (WGA) but with different specificity to each. For patients with AD, the C/W ratio is typically above 0.95 and the percentage of BuChE unbound to Con A relative to the total BuChE is at least about eight percent (8%).

In an alternative embodiment of the invention there is provided a monoclonal antibody specific for BuChE with an altered glycosylation pattern used to detect its presence.

The body fluid analysed can be cerebrospinal fluid (CSF), blood or blood plasma. Advantageously, when said body fluid is blood, blood plasma is prepared from the blood for analysis.

According to a further aspect of the present invention there is provided an abnormal isoform of BuChE with an altered pattern of glycosylation and characterised in that it has a relatively lesser affinity for Concanavalin (Con A) than BuChE with an unaltered glycosylation pattern.

According to another aspect of the present invention there is provided an abnormal isoform of BuChE with an altered glycosylation pattern and characterised in that it has a relatively lesser affinity for Con A than BuChE with an altered glycosylation pattern.

### BRIEF DESCRIPTION OF THE DRAWINGS

FIG. 1 shows the correlation between the level of AChE and BChE in cerebrospinal fluid. (Black squares=controls. Open circles=AD.) This figure shows that there is a positive correlation in the levels of AChE and BChE suggesting that similar biochemical mechanisms may underly the decrease in activity of both enzymes in AD CSF.

FIG. 2 is a plot of the AChE C/W ratio vs. the percentage (%) BChE unbound to Con. A. This figure shows that there is no clear correlation between these analytes. This figure also shows that by combining both measures, almost complete separation can be achieved between the AD and control groups.

FIG. 3 is a plot of the percentage (%) BChE unbound to Con A vs. age (yr) for both AD and controls. This figures shows that there is no relationship between BChE glycosylation and age. Thus disease status is the only correlate.

FIG. 4 is a three dimensional plot of the total BChE, C/W ratio and the percentage (%) BChE unbound to Con A showing complete separation of the AD and control groups.

### BEST MODE FOR CARRYING OUT THE INVENTION

AChE, acetylcholinesterase; butyrylcholinesterase (BuChE) ChE, cholinesterase; Aβ, amylid β protein; AD, Alzheimer's disease; DP, diffuse plaques; ND, other neurological diseases; PMI, post mortem interval; PBS, phosphate-saline buffer; TB, Tris buffer; TSB, Tris-saline buffer; SS, salt-soluble supernatant; TS, Triton X-100-soluble supernatant; AF, amphiphilic fraction; HF, hydrophilic fraction; G^{a}, globular amphiphilic isoform; G^{na}, globular non-amphiphilic isoform; and agglutinins from Canavalia ensiformis (Concanavalin A), Con A; Triticum vulgaris (wheat germ), WGA; Ricinus communis, RCA₁₂₀; Lens culinaris, LCA; Dolichus biflorus, DBA; Ulex europaeus, UEAₗ; Glycine max, SBA; and Arachis hypogaea, PNA.

Immobilised lectins (Con A- and LCA-Sepharose, WGA-, RCA₁₂₀-, DBA-, UEAₗ; SBA and PNA-agarose), phenylagarose, bovine liver catalase, E. coli alkaline phosphatase, polyoxyethylene-10-oleyl ether (Brij 97), Triton X-100, tetraisopropyl pyrophosphoramide (iso-OMPA), 1,5-bis(4-allydimethyl-ammoniumphenyl)-pentan-3-1 dibromide (BW284c51), acetylthiocholine iodide and 5,5′-dithio-bis-2-nitrobenzoic acid (DTNB) were all obtained from Sigma-Aldrich Pty. Ltd. (Seven Hills, NSW, Australia). Sepharose CL-4B was purchased from Pharmacia Biotech AB (Uppsala, Sweden).

TABLE 1
Binding of CSF BChE to Various Lectins

% BChE Unbound to Lectin

| Lectin | Controls | AD | DNAT | OND |
|---|---|---|---|---|
| Con A | 3.0 ± 0.6 | 6.4 ± 0.7* | 2.0 ± 0.7 | 2.4 ± 0.9 |
| LCA | 57.4 ± 6.2 | 29.4 ± 2.2 | 41.0 ± 1.5 | 39.6 ± 3.1 |
| WGA | 10.4 ± 3.7 | 6.2 ± 0.8 | 4.6 ± 0.9 | 4.4 ± 1.0 |
| RCA | 39.7 ± 4.0 | 38.5 ± 2.7 | 32.0 ± 9.4 | 42.3 ± 3.7 |
| DBA | 98.0 ± 1.2 | 93.8 ± 1.9 | 100.0 ± 0.0 | 83.8 ± 10.5 |
| PNA | 86.6 ± 6.1 | 93.3 ± 1.5 | 92.7 ± 1.5 | 95.8 ± 4.3 |
| SBA | 83.7 ± 2.1 | 86.9 ± 1.5 | 96.0 ± 1.0 | 91.0 ± 2.4 |
| UEAₗ | 92.1 ± 1.6 | 90.4 ± 1.7 | 91.3 ± 0.3 | 93.0 ± 3.0 |

*=significantly different from controls (P = 0.002).

The following Examples relate to experiments conducted with acetylcholinesterase (AchE). One skilled in the art would readily be able extrapolate from the following Examples to make a diagnostic test for Alzheimer's Disease comprising the detection of butyrylcholinesterase (BchE) with an altered glycosylation pattern

### EXAMPLE 1

### Lectin Binding AChE Experiments in AD Patients

Lumbar or ventricular CSF was obtained post mortem; 18 controls with no clinical or pathological dementia and no clinical or pathological dementia and no evidence of brain pathology, 27 cases of AD, 7 cases of dementia non-AD type (DNAT, 5 frontal lobe dementia, 1 Lewy body dementia/Parkinson's disease and 1 multi-infarct dementia/congophilic amyloid angiolpathy), and 6 cases of other neurological disorders (ND, 4 Huntington's disease, 1 schizophrenia and 1 corticobasal degeneration). The average age in the control group was 68±4 years, there were 10 females and 8 males and the PMI was 40±6. In the AD group the age was 81±2 years, there were 13 female and 14 males and the PMI was 35±6. In the ND group the age was 65±6, there were 3 females and 3 males and the PMI was 45±12. In the DNAT group the age was 76±3, there were 4 female and 3 males and the PMI was 34±11. Samples of CSF were stored at −70° C. and centrifuged at 1,000× g for 15 min prior to analysis. AChE activity was assayed at 22° C. by a modified microassay of the Ellman method (Ellman et al. 1961). Aliquots (0.3 ml) were mixed with 0.1 ml of Sepharose 4B in PBS (control), Concanavalin A (Con A) or wheat germ agglutinin (WGA, Triticum vulgaris) immobilised on Sepharose. The enzyme-lectin mixture was incubated overnight at 4° C., and then centrifuged (1,000× g, 15 min). AChE activity was assayed in the supernatant fractions. Data were analysed using a Student's t-test.

The total AChE values in ventricular CSF samples of subjects=>60 yrs old were significantly lower in the AD group (6.98±0.82 nmol/min/ml) than in controls (17.24±4.28 nmol/min/ml; P<0.001). However, as reported previously, (Appleyard et al., 1983), the large overlap (40%) between the data prevents the use of total AChE as a significant diagnostic marker.

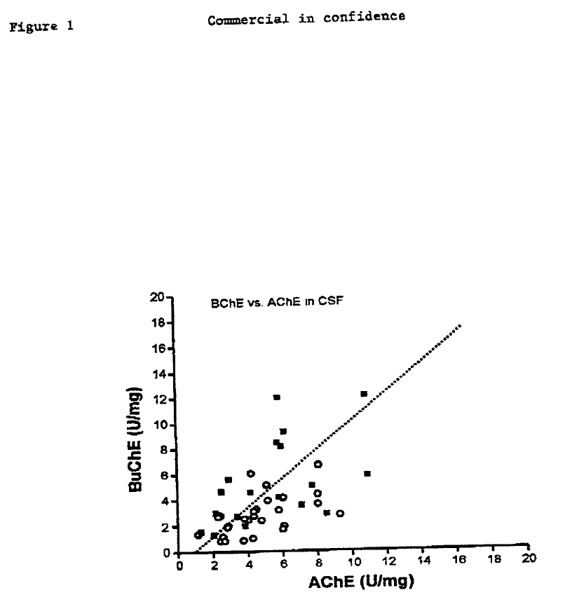

However, lectin-binding analysis revealed a significant difference between the AD group and controls. Approximately 75-95% of the AChE in the CSFs bound to Con A or WGA. A ratio (C/W ratio) was defined as AChE unbound to Con A divided by AChE unbound to WGA. The mean C/W ratio for the AD group was significantly different from controls (FIG. 1). Of the 27 CSFs from confirmed AD, 21 samples had a C/W ratio>0.95. All 18 control samples had C/W<0.95, without significant differences between younger (n=5, C/W=0.37±0.10) and older subjects (n=6, 0.38±0.08) samples. No correlation in C/W ratio was noted with post mortem interval (PMI). The data are represented graphically in FIG. 1.

The data indicate that lectin-binding analysis of CSF AChE could provide a diagnostic test for AD which is 80% sensitive and 97% specific. Thus it was proposed that differences observed in the glycosylation pattern of AChE in CSF may be useful as an ante mortem diagnostic marker for AD, particularly when used in combination with measurement of other biochemical markers.

### EXAMPLE 2

### Human Brain and CSF Samples for AChE Experiements

Ventricular and lumbar CSF, frontal cortical and cerebellar samples were obtained post mortem and stored at −80° C. Three non-AD groups of samples were defined, 1) controls with no clinical or pathological features of dementia (n=18), 2) individuals who showed no clinical signs of dementia but who were found to have a moderate number of non-neuritic Ab-immunoreactive diffuse plaques (DP), but no evidence of neocortical neurofibrillary changes (n=6), and 3) individuals with various neurological diseases (ND) containing 7 cases of non-AD type dementia (5 frontal lobe dementia, 1 Lewy body dementia and 1 vascular dementia) and 7 cases of other neurological disorders (4 Huntington's disease, 1 Parkinson's disease, 1 schizophrenia and 1 corticobasal degeneration). Cases of AD were selected on the basis of their clinical history of dementia and neuropathological CERAD diagnosis (Mirra et al., 1994). All the CSF samples included in the AD and ND groups were ventricular and only 5 control and 1 DP CSF samples (from a total of 18 and 6 subjects, respectively) were taken by lumbar puncture. Immunohistochemical examination of the cerebellar samples showed that, unlike the frontal cortex, none of the AD tissue possessed compact neuritic amyloid plaque deposition (data not shown), consistent with previous studies (Mann etal., 1996).

It has been shown (Grass) et al., 1982; Fishman et al., 1986; Sáez-Valero etal., 1993) that for a post mortem interval (PMI) greater than 72 hr, storage at −20° C. or repeated cycles of freeze-thawing caused degradation of AChE, which confounded glycosylation analysis. Therefore, only samples with a PMI of less than 72 hr (PMI=36±4 hr) were used. There was no significant difference in PMI between each group of samples.

Preparation of Samples and Extraction of AChE

Samples of CSF were thawed slowly at 4° C. and then centrifuged at 1,000× g for 15 min prior to use. Small pieces (0.5 g) of frontal cortex and cerebellum were thawed slowly at 4° C., weighed and homogenised (10% w/v) in ice-cold Tris-saline buffer (TSB; 50 mM Tris-HCl, 1 M NaCl, and 50 mM MgCl₂, pH 7.4) containing a cocktail of proteinase inhibitors (Silman et al., 1978). Tissues were homogenised with a glass/Teflon homogeniser and then sonicated with 10-15 bursts at 50% intermittency at setting 4 using a Branson sonifier. The suspension was centrifuged at 100,000× g at 4° C. in a Beckman L8-80M ultracentrifuge using a 70.1 Ti rotor for 1 hr to recover a salt-soluble ChE fraction (SS). The pellet was re-extracted with an equal volume of TSB containing 1% (w/v) Triton X-100, and the suspension centrifuged at 100,000× g at 4° C. for 1 hr to obtain a Triton X-100-soluble ChE fraction (TS). This double-extraction method recovered 80-90% of the total ChE activity (SaezValero et al., 1993; Moral-Naranjo et al., 1996).

AChE Assay and Protein Determination

AChE activity was determined by a modified microassay method of Ellman (Sáez-Valero et al., 1993). One unit of AChE activity was defined as the number of nmoles of acetylthiocholine hydrolysed per min at 22° C. Protein concentrations were determined using the bicinchoninic acid method with bovine serum albumin as standard (Smith et al., 1985).

Hydrophobic Interaction Chromatography on Phenyl-agarose

Amphiphilic AChE forms were separated from hydrophilic forms by hydrophobic interaction chromatography on phenyl-agarose as previously described (Sáez-Valero et al., 1993). CSF (10 ml-pooled from four samples obtained from four different subjects) was applied to a column (10×1 cm) of phenyl-agarose. A hydrophilic fraction (HF) containing hydrophilic isoforms of AChE was eluted with 30 ml of TSB, and then an amphiphilic fraction (AF) containing bound amphiphilic isoforms was eluted with 50 mM Tris-HCl (TB, pH 7.4) containing 2% (w/v) Triton X-100. Peak fractions with high AChE activity were pooled and concentrated using Ultrafree-4 Centrifugal Filter Device Biomax 10 kDa concentrators (Millipore Corporation, Bedford, Mass., USA).

Sedimentation Analysis

Molecular isoforms of AChE were analysed by ultracentrifugation at 150,000× g in a continuous sucrose gradient (5-20% w/v) for 18 hr at 4° C. in a Beckman SW40 rotor. The gradients contained 10 ml of 50 mM Tris-HCl (pH 7.4) containing 0.5 M NaCl, 50 mM MgCl₂ and 0.5% (w/v) Brij 97. Approximately 40 fractions were collected from the bottom of each tube. Enzymes of known sedimentation coefficient, bovine liver catalase (11.4S, S_{20,w} Svedberg Units) and E. coli alkaline phosphatase (6.1S) were used in the gradients to determine the approximate sedimentation coefficients of AChE isoforms. A ratio of AChE species G_{4/}(G₂+G₁), that reflected the proportion of G₄ molecules (G₄^{na}+G₄^{a}) versus both light globular AChE isoforms, G₂^{a} and G₁^{a} was defined. Estimation of the relative proportions of each molecular form of AChE was performed by adding the activities under each peak (G₄ or G₂+G₁) and calculating the relative percentages (recovery>95%).

Lectin-binding Analysis of AChE

Samples (0.3 ml) were added to 0.1 ml (hydrated volume) of Sepharose 4B (control), Con A, WGA, RCA₁₂₀, LCA, DBA, UEAₗ, SBA or PNA immobilised in agarose or Sepharose. The enzyme-lectin mixture was incubated overnight at 4° C. with gentle mixing. Bound and free AChE were separated by centrifugation at 1000× g for 15 min at 4° C. in a Beckman J2-21 M/E centrifuge using a JA-20 rotor, and the unbound AChE was assayed in the supernatant fraction. Percentage of unbound AChE in the lectin incubation was calculated as (AChE unbound to lectin/AChE unbound to Sepharose)×100. The C/W ratio was calculated according to the formula, AChE activity unbound in the Con A incubation divided by the AChE activity unbound in the WGA incubation. It was observed that this ratio detects a specific alteration in AChE glycosylation that occurs in AD CSF.

Lectin Binding of CSF AChE

To examine the glycosylation of AChE, CSF samples from 18 controls and 30 cases of AD were incubated with different immobilised lectins, which recognise different sugars. AChE bound strongly to Con A, WGA and LCA but weakly to RCA₁₂₀, PNA, DBA, UEA, and SBA (Table 1), suggesting that most of the enzyme was devoid of terminal galactose, terminal N-acetyl-galactosamine or fucose.

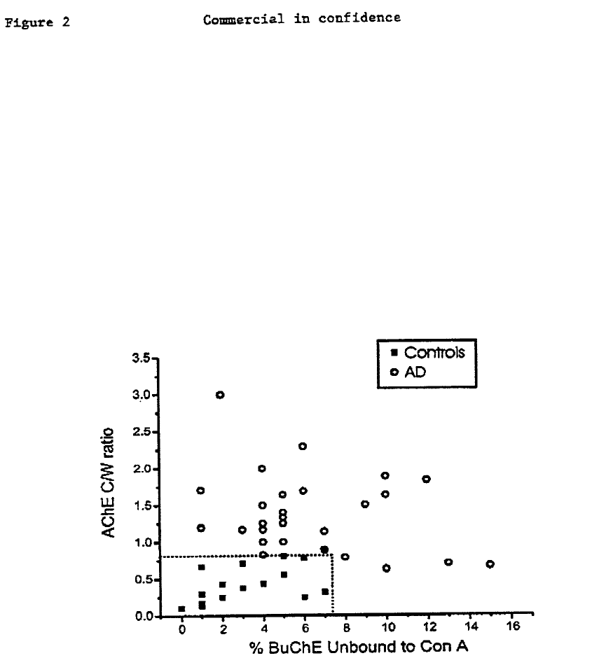

There was a small but significant difference in the binding of AChE to Con A and WGA between the AD group and controls (Table 1). As the percentage of AChE unbound in the AD CSF was increased for Con A and decreased for WGA, a ratio (C/W=[% AChE that does not bind to Con A]/[% AChE that does not bind to WGA]) was defined, which provided greater discrimination between the two groups (Table 1). Using this method, it was found that the mean C/W ratio for the AD group was significantly greater than for the other control groups, including cases with diffuse plaques (non-demented, DP), and patients with other neurological and neuropsychiatric diseases (ND) (FIG. 2), consistent with the results shown in Example 1. Of the 30 CSF samples from confirmed AD cases, 24 samples were above a cut-off value of C/W=0.95 (FIG. 2). Only one sample from 18 controls, one out of 6 samples from cases with diffuse plaques, and one out of 14 samples from the other neurological diseases group, a frontal lobe dementia case, were above this value. The 6 AD samples with C/W ratios lower than 0.95 had C/W ratios>0.60, a value higher than the C/W mean of the non-AD groups (control=0.53±0.1; DP=0.46±0.2; ND=0.53±0.1).

No correlation could be found between the C/W ratio and the PMI that could suggest that different C/W ratio in the AD group was due to differences in PMI. Furthermore, there was no significant difference in the PMI between the AD (33±6 hr) and non-AD samples (40±6 hr).

CSF samples were additionally analysed for total AChE activity (FIG. 2). As previously reported (Appleyard et al., 1983; Atack et al., 1988), the CSF from patients with AD had significantly lower AChE activity (6.5±0.8 U/ml) than controls (15.8±2.9 U/ml) or patients with other diseases (12.4±2.4 U/ml). However, the C/W ratio was a more reliable index of clinical status than the total level of AChE activity in the CSF (FIG. 2).

AChE Isoforms in CSF

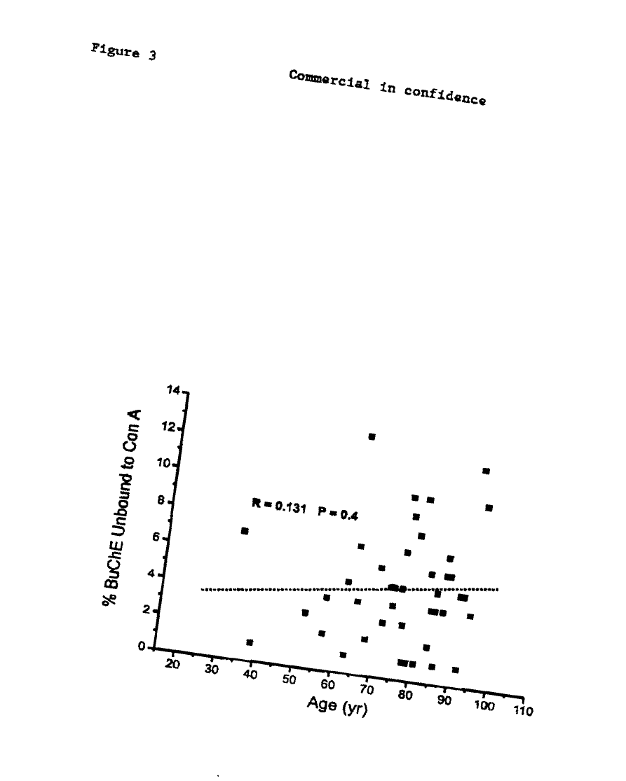

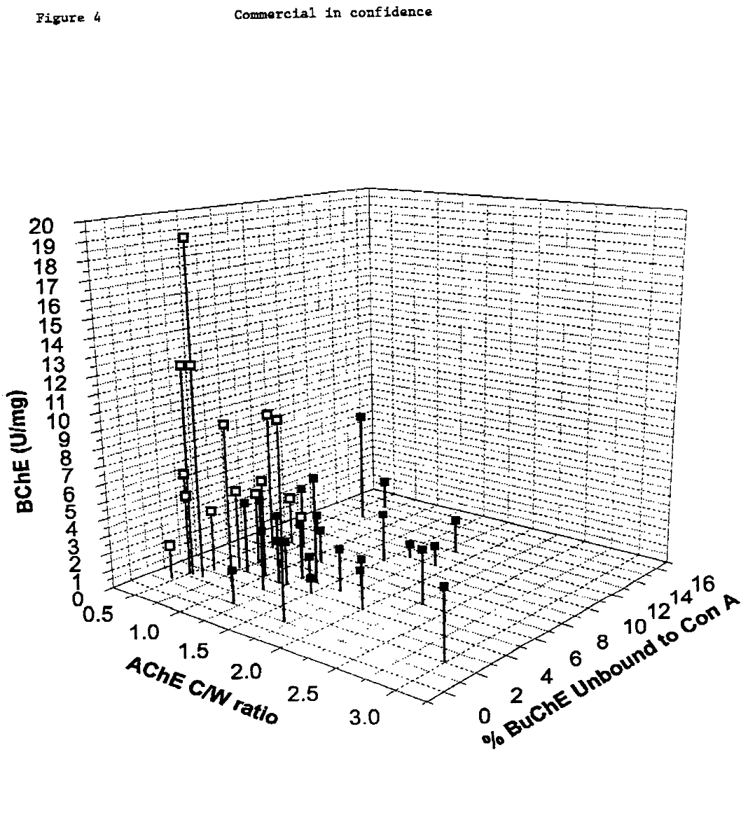

To determine whether the alteration in glycosylation was due to changes in a specific isoform of AChE, CSF samples were analysed by hydrophobic interaction chromatography to separate amphiphilic (G^{a}) and hydrophilic species (G^{na}) (FIG. 3), and by sucrose density gradient centrifugation in 0.5% (w/v) Brij 97 to separate individual molecular weight isoforms (G₄,G₂ and G₁) (FIG. 3). A decrease in the proportion of G₄, AChE in AD CSF compared to controls (FIG. 4, top panels) was observed. The ratio of (G₄,/(G₂+G₁) was significantly (P<0.01) higher in controls (1.80±0.12; n=4) than in AD cases (1.16±0.12; n=4). To separate hydrophilic isoforms from amphiphilic isoforms, CSF was fractionated by hydrophobic interaction chromatography on phenyl-agarose (FIG. 3). A smaller percentage of AChE in the normal CSF bound to phenyl agarose (12±3%, n=4) than in the AD CSF (38±4%, n=4; P<0.001). Sedimentation analysis of the unbound hydrophilic fraction (HF) showed a main peak of 10.8S, consistent with a hydrophilic tetrameric (G₄^{na}) isoform (Atack et al., 1987), as well as a small amount of lighter AChE isoforms, 5.1S dimers and 4.3S monomers (FIG. 4). The bound amphiphilic fraction from the phenyl-agarose column contained a minor peak of 9.0-9.5S (probably an amphiphilic tetramer, G₄^{a} and a major peak of amphiphilic globular dimer (G₂^{a}, 4.2S) and monomer (G₁^{a}, 3.1S). The level of the amphiphilic light isoforms was greater in the AD CSF than in controls (FIG. 4).

Glycosylation of Individual AChE Isoforms in CSF

Incubation of the HF and AF with immobilised Con A and WGA showed that there was an increase in the C/W ratio in AD CSF, and that the high CIW ratio was associated with an amphiphilic fraction containing dimers and monomers (FIG. 4). The data indicate that the contribution of G₂ and G₁ AChE in AD CSF was mainly responsible for the increased C/W ratio of total AChE in the AD CSF.

Levels of AChE in Frontal Cortex and Cerebellum

To determine whether the changes in AChE glycosylation reflect a change in the expression or glycosylation of brain AChE isoforms, the levels of AChE activity in samples of frontal cortex and cerebellum were examined. Samples were homogenised with salt and Triton X-100 to extract soluble and membrane-bound AChE isoforms, and then the AChE activity determined in both fractions (Table 2). The frontal cortex samples from AD patients had significantly less AChE activity in the Triton X-100-soluble (TS) fraction (˜40%), with no difference in levels in the salt-soluble (SS) fraction compared with controls (Table 3). The results are consistent with previous studies that indicate that the major G₄ isoform is decreased only in the TS fraction (Younkin et al., 1986; Seik et al., 1990). A small but significant decrease (˜15%) in the protein content of the TS fraction of both AD and ND groups was also observed. The level of AChE in the frontal cortex samples of the ND group was significantly different from controls in both the SS and TS fraction (Table 2). However, as the ND group was heterogeneous (2 frontal lobe dementia, 1 Huntington's disease and 1 Parkinson's disease), the significance of changes in AChE levels is unclear. Levels of AChE in cerebellum were also significantly decreased in the TS fraction from the AD group (Table 2).

Glycosylation of AChE in Frontal Cortex and Cerebellar

To determine whether different glycosylation pattern of AChE in AD CSF is also present in the AD brain, the glycosylation of brain AChE was examined by lectin binding. Homogenates from frontal cortex and cerebellum were incubated with immobilised Con A or WGA and the amount of activity unbound was calculated. In the AD frontal cortex, the % AChE activity that did not bind to Con A or WGA was significantly different from controls (Table 3). Similar to the CSF AChE, the C/W ratio of frontal cortex AChE was greater in AD than in non-AD samples (Table 3). This increase was due to a large increase in the amount of AChE that did not bind to Con A, and was in spite of an increase in the amount of AChE that did not bind to WGA (Table 3). There was no increase in the C/W ratio in the DP and ND group (Table 3). No difference in lectin binding was observed between AD and non-AD groups in the cerebellar fractions (Table 3.)

AChE Isoforms in Frontal Cortex and Cerebellum

To determine the cause of the altered glycosylation in AD brain, the pattern of AChE isoforms in the frontal cortex and cerebellum was examined. Equal volumes of SS and ST supernatants (total AChE activity) were pooled and then analysed by sucrose density gradient sedimentation with 0.5% (w/v) Brij 97 to separate the major AChE isoforms (FIG. 5). Based on their sedimentation coefficients (Atack et al., 1986; Massoulié et al., 1982) it was possible to identify hydrophilic (G₄^{na}, 10.7±0.1S) and amphiphilic tetramers (G₄^{a}, 8.6±0.1S) amphiphilic dimers (G₂^{a}, 4.7±0.1S) and monomers (G₁^{a}, 3.0±0.1S) of AChE (FIG. 6). There were no differences in the sedimentation coefficient (S) of individual isoforms from each group. Due to the overlap in the sedimentation coefficients between AChE G₄^{na} and G₄^{a}, it was not possible to separate these isoforms completely (FIG. 5). However, the contribution of G₄^{a} was greater than G₄^{na}. Asymmetric (A₁₂) AChE isoforms were identified in trace amounts (2-5%) in some of the fractions.

A significant decrease in G₄, (40% of the mean control value, P<0.001) and in G₂+G₁ AChE (60% of the mean control value, P=0.002) was detected in the fractions from AD frontal cortex. This change in the relative proportion of AChE isoforms was reflected in the G₄/(G₂+G₁) ratio, which was significantly lower in the AD samples (Table 3). Interestingly, a similar and statistically significant decrease was found in the G₄/(G₂+G₁) ratio for the DP subjects. This change in ratio was due to a 25% increase in the level of G₂+G₁ and a small decrease (10%) in G₄ AChE, although neither change on its own was statistically significant. No variation in AChE G₄/(G₂+G₁) was found in the AD cerebellum (Table 3), despite a statistically significant decrease (40%) in AChE in the TS fraction (Table 2) and in the total level of G₄ AChE (G₄ in controls=380±40 U/ml, G₄ in ADs=195±70 U/ml; P=0.008).

Glycosylation of Individual AChE Isoforms in Frontal Cortex and Cerebellum

Since it was found that the ratio of AChE was altered in the frontal cortex of AD patients, steps were taken to ascertain whether the increase in the C/W ratio of brain AChE was due to a change in glycosylation or in the expression of a specific isoform of AChE. Individual AChE isoforms were separated by sucrose gradient centrifugation and then fractions from the G₄ or G₂+G₁ peaks were pooled, dialysed against TSB-Triton X-100 buffer and concentrated by ultrafiltration. AChE isoforms were then assayed by lectin binding and a C/W ratio calculated for each isoform (FIG. 5).

No differences were observed in the C/W ratio of G₄, AChE between the AD and non-AD groups (FIG. 5). However, in all frontal cortex samples the G₂+G₁ fraction possessed C/W ratios>1.00, demonstrating that G₂ or G₁ AChE is glycosylated differently from the G₄ isoform. Moreover, the C/W ratio for G₂+G₁ AChE was higher in the AD group than controls or DP. Similarly, the C/W ratio of the amphiphilic fraction from CSF (containing predominantly G₂+G₁ AChE) was higher in the AD group than in controls (FIG. 3). There was no correlation between the G₄/(G₂+G₁) ratio and the C/W ratio in the DP group in frontal cortex. In the cerebellum, no differences were observed in the C/W ratios of G₄ AChE or G₂+G₁ AChE between AD and non-AD groups (FIG. 4). The G₂+G₁ fractions, from both AD and non-AD cerebellar groups, had a C/W<0.50, in contrast to the same fraction from frontal cortex (C/W>1.00) indicating differences in the pattern of glycosylation of G₂+G₁ AChE between both brain areas.

This example shows that AChE is glycosylated differently in the frontal cortex and CSF of AD patients compared with AChE from non-AD groups including patients with non AD-type dementias. This difference in glycosylation is due to an increase in the proportion of differentially glycosylated amphiphilic dimeric and monomeric AChE in the AD samples. The results suggest that the abnormally glycosylated AChE in AD CSF may be derived from the brain as a similar difference in glycosylation was also found in the frontal cortex of AD patients.

TABLE 1
Lectin-binding of AChE in CSF.

AChE unbound (%)

| Lectin | Control | AD |
|---|---|---|
| Con A | 5.5 ± 0.8 | 10.0 ± 1.1^{b} |
| WGA | 11.3 ± 1.7 | 7.0 ± 0.6^{b} |
| Con A/WGA | 0.53 ± 0.1 | 1.37 ± 0.1^{a} |
| (C/W) | | |
| LCA | 17.2 ± 4.2 | 15.0 ± 1.3 |
| RCA₁₂₀ | 74.1 ± 3.4 | 70.8 ± 2.7 |
| SBA | 83.0 ± 2.1 | 82.2 ± 1.9 |
| UEAₗ | 91.6 ± 2.2 | 87.6 ± 1.9 |
| PNA | 92.4 ± 1.7 | 92.3 ± 1.4 |
| DBA | 98.9 ± 0.8 | 95.8 ± 1.7 |

All the CSFs were taken post mortem and the diagnosis confirmed by pathological examination. CSF from normal subjects (Control group: n=18; 67±4 years at death; 11 Females/7 Males) and AD patients (AD group: n=30; 79±2 y; 15F/15M) were incubated either with an equal volume of the different immobilized lectins, and then centrifuged. AChE was assayed in the supernatant fractions. The data represent the means±SEM.

TABLE 2
AChE activity and protein levels in human frontal cortex and cerebellum

| | AChE activity | | Protein | |
|---|---|---|---|---|
| | (U/ml) | | (mg/ml) | |
| Group/Source | SS | TS | SS | TS |
| Control | | | | |
| Frontal Cortex | 3.7 ± 0.4 | 15.1 ± 1.5 | 2.1 ± 0.1 | 2.4 ± 0.1 |
| (n = 11; 63 ± 5 y; | | | | |
| 7F/4M) | | | | |
| Cerebellum | 64 ± 6 | 264 ± 25 | 2.5 ± 0.1 | 1.9 ± 0.1 |
| (n = 7; 66 ± 5 y; | | | | |
| 4F/3M) | | | | |
| DP | | | | |
| Frontal Cortex | 5.5 ± 0.9 | 12.7 ± 1.7 | 2.1 ± 0.1 | 2.2 ± 0.1 |
| (n = 6; 81 ± 2 y; | | | | |
| 4F/2M) | | | | |
| Cerebellum | 49 ± 8 | 182 ± 46 | 2.6 ± 0.1 | 1.9 ± 0.1 |
| (n = 5; 81 ± 3 y; | | | | |
| 3F/2M) | | | | |
| ND | | | | |
| Frontal Cortex | 5.4 ± 0.6^{a} | 9.3 ± 1.7^{b} | 2.1 ± 0.2 | 2.0 ± 0.1^{b} |
| (n = 4; 67 ± 9 y; | | | | |
| 2F/2M) | | | | |
| Cerebellum | 45 ± 8 | 160 ± 50 | 2.7 ± 0.2 | 2.3 ± 0.2 |
| (n = 2; 78 ± 14 y; | | | | |
| 1F/1M) | | | | |
| AD | | | | |
| Frontal Cortex | 3.7 ± 0.3 | 9.0 ± 0.9^{a} | 2.1 ± 0.1 | 2.1 ± 0.1^{a} |
| (n = 14; 73 ± 3 y; | | | | |
| 8F/6M) | | | | |
| Cerebellum | 48 ± 12 | 160 ± 28^{b} | 2.6 ± 0.1 | 2.0 ± 0.1 |
| (n = 7; 73 ± 6 y; | | | | |
| 5F/2M) | | | | |

Tissue from frontal cortex or cerebellum was homogenized and salt-soluble (SS) and Triton X-100-soluble (TS) extracts obtained. The extracts were then assayed for AChE and protein. DP=non-demented subjects with diffuse plaques; ND=individuals with other neurological diseases and dementias of non-AD type; AD=individuals with Alzheimer's disease. F=female; M=male; y=age in years. Values are means±SEM.

TABLE 3
Lectin binding and AChE isoforms in frontal cortex and cerebellum

| | Lectin binding | | | |
|---|---|---|---|---|
| | AChE | AChE | | |
| | unbond to | unbound | AChE ratio | |
| Group/Source | Con A (%) | to WGA (%) | C/W | G₄/ (G₂+G₁) |
| Control | | | | |
| Frontal Cortex | 6.9 ± 0.8 | 12.3 ± 1.2 | 0.56 ± 0.03 | 1.90 ± 0.14 |
| (n = 11; 63 ± 5 | | | | |
| y; 7F/4M) | | | | |
| Cerebellum | 1.8 ± 0.1 | 10.7 ± 0.9 | 0.18 ± 0.02 | 3.02 ± 0.14 |
| (n = 7; 66 ± 5 | | | | |
| y; 4F/3M) | | | | |
| DP | | | | |
| Frontal Cortex | 7.4 ± 0.8 | 15.0 ± 1.0 | 0.50 ± 0.06 | 1.32 ± 0.12^{b} |
| (n = 6; 81 ± 2 | | | | |
| y; 4F/2M) | | | | |
| Cerebellum | 2.9 ± 0.7 | 12.2 ± 1.3 | 0.23 ± 0.05 | 2.18 ± 0.33 |
| (n = 5; 81 ± 3 | | | | |
| y; 3F/2M) | | | | |
| ND | | | | |
| Frontal Cortex | 7.0 ± 0.6 | 13.2 ± 1.2 | 0.47 ± 0.05 | 2.61 ± 0.73 |
| (n = 4; 67 ± 9 | | | | |
| y; 2F/2M) | | | | |
| Cerebellum | 1.8 ± 0.2 | 10.1 ± 0.3 | 0.21 ± 0.10 | 2.50 ± 0.70 |
| (n = 2; 78 ± 14 | | | | |
| y; 1F/1M) | | | | |
| AD | | | | |
| Frontal Cortex | 13.1 ± 1.3^{a} | 19.7 ± 1.4^{a} | 0.66 ± 0.03^{b} | 1.34 ± 0.18^{b} |
| (n = 14; 73 ± 3 | | | | |
| y; 8F/6M) | | | | |
| Cerebellum | 2.4 ± 0.3 | 13.5 ± 2.3 | 0.19 ± 0.02 | 2.33 ± 0.49 |
| (n = 7; 73 ± 6 | | | | |
| y; 5F/2M) | | | | |

SS and TS fractions from frontal cortex and cerebellum were pooled in equal volumes and then analyzed by lectin binding using immobilized Con A and WGA. The C/W ratio was calculated as defined in Table 2. Aliquots of the supernatants (SS+TS) were also analyzed by sucrose density gradient sedimentation to identify AChE isoforms. Values are means±SEM.

### REFERENCES

The following references are incorporated herein by reference:

Appleyard M. B. and McDonald B. (1992) Acetylcholinesterase and butyrylcholinesterase activities in cerebrospinal fluid from different levels of the neuraxis of patients with dementia of the Alzheimer type. J. Neurol. Neurosurg. Psychiat 55, 1074-1078.

Appleyard M. E., Smith A. D., Berman P., Wilcock G. K., Esiri M. M., Bowen D. M. and Neary D. (1987) Cholinesterase activities in cerebrospinal fluid of patients with senile dementia of the Alzheimer Type. Brain 110, 1309-1322.

Appleyard M. E., Smith A. D., Wilcock G. K. and Esiri M. M. Decreased CSF acetylcholinesterase activity in Alzheimer's disease. Lancet 1983; 20:452.

Arendt T., Bigl V., Walther F. and Sonutag M. (1984) Decreased ratio of CSF acetylcholinesterase to butyrylcholinesterase activity in Alzheimer's disease. Lancet i, 173.

Arendt T., Bruckner M. K., Lange M. and Bigl V. (1992) Changes in acetylcholinesterase and butyrylcholinesterase in Alzheimer's disease resemble embryonic development—A study of molecular forms. Neurochem. Intl. 21, 381-396.

Atack J. R., Perry E. K, Bonham, J. R., Candy, J. M., and Perry R. H. (1986) Molecular forms of acetylcholinesterase in the aged human central nervous system. J. Neurochem. 47, 267.

Atack J. R., Perry E. K., Bonham J. R., and Perry R. H. (1987) Molecular forms of acetylcholinesterase and butyrylcholinesterase in human plasma and cerebrospinal fluid. J. Neurochem, 48, 1845-1850.

Atack J. R., May C., Kaye J. A., Kay A. D., and Rapoport S. I. (1988) Cerebrospinal fluid cholinesterases in aging and in dementia of the Alzheimer type. Ann. Neurol. 23,161-167.

Atack J. R., Perry E. K., Bonham J. R., Perry R. H., Tomlinson B. E., Blessed G. and Fairbairn A. (1983) Molecular forms of acetylcholinesterase in senile demential of Alzheimer's type: selective loss of the intermediate (10S) form. Neurosci. Lett. 40, 199-204.

Atack J. R., Perry E. K., Perry R. H., Wilson I. D., Bober M. J., Blessed G. and Tomlinson B. E. (1985) Blood acetyl and butyrylcholinesterase in senile dementia of Alzheimer type. J. Neurol. Sci. 70,1-12.

Blass J. P., Blennow K., Delacourte A., Frisoni G. B., Jefferies W. A., McRae A., Wisniewski H. M., Parshad R., Scinto L. F. M., Scheltens P., Riekkinen P. J., Swanwick G. R. J., Wahlund L. O., Trojanowski J. Q., Winbland B., Ihara Y, et al. (1998)

Consensus report of the Working Group on: “molecular and biochemical markers of Alzheimer's disease”. Neurobiol. Aging 19, 109-116.

Davies C. A., Mann D. M. A., Sumpter P. Q., and Yates P. O. (1987) A quantitative morphometric analysis of the neuronal and synaptic content of the frontal and temporal cortex in patients with Alzheimer's disease. J. Neurol. Sci, 78, 151-164.

Ellman G. E., Courtney K. D., Andres Jr. V. and Featherstone R. M. (1961) A new and rapid calorimetric determination of acetylcholinesterase activity. Biochem. Pharmacol. 7, 88-95.

Fishman E. B., Siek G. C., MacCallum R. D., Bird E. D., Volicer L., and Marquis J. K. (1986) Distribution of the molecular forms of acetylcholinesterase in human brain, alterations in dementia of the Alzheimer type. Ann. Neurol. 19, 246-252.

Friede R. L. (1965) Enzyme histochemical studies of senile plaques. J. Neuropathol. Exp. Neurol. 24, 477-491.

Geula C., and Mesulam M. M. (1989) Special properties of cholinesterases in the cerebral cortex of Alzheimer's disease. Brain Res. 498, 185-189.

Grassi J., Viguy M., and Massoulié J. (1982) Molecular forms of acetylcholinesterase in bovine caudate nucleus and superior cervical ganglion: solubility properties and hydrophobic character. J. Neurochem. 38, 457-469.

Guillozet A. L., Smiley J., Mash D. C. and Mesulam M. M. (1997) Butyrylcholinesterase in the life cicle of amyloid plaques. Ann. Neurol. 42, 909-918.

Hogan B., Costantini F., and Lacy E. (1986) Manipulating the Mouse Embryo, A laboratory manual, Cold Spring Habor, N.Y.

Inestrosa N. C., Alvarez A., Perez C. A., Moreno R. D., Vicente M., Linker C., Casanueva O. I., Soto C., and Garrido J. (1996a) Acetylcholinesterase accelerates assembly of amyloid-b-peptides into Alzheimer's fibrils—possible role of the peripheral site of the enzyme. Neuron 16, 881-891.

Inestrosa N. C., Alvarez A., and Calderon F. (1996b) Acetylcholinesterase is a senile plaque component that promotes assembly of amyloid beta-peptide into Alzheimer's filaments. Mo 1. Psychiatry 1, 359-361.

Kang J., Lemaire H. G., Unterbeck A., Salbaum J. M., Masters C. L., Grzeschik K. H., Multhaup G., Beyreuther K., and Müller-Hill B. (1987) The precursor of Alzheimer's disease amyloid A4 protein resembles a cell-surface receptor. Nature 325, 733-736.

Liao J., Heider H., Sun M. C., and Brodbeck U. (1992) Different glycosylation in acetylcholinesterases from mammalian brain and erythrocytes. J. Neurochem. 58, 1230-1238.

Luo Z., Fuentes M. E., and Taylor P. (1994) Regulation of acetylcholinesterase mRNA stability by calcium during differentiation from myoblasts to myotubes. J. Biol. Chem. 269, 27216-27223.

Mann D. M. A., Iwatsubo T., and Snowden J. S. (1996) Atypical amyloid (Ab) deposition in the cerebellum in Alzheimer's disease: an immunohistochemical study using end-specific Ab monoclonal antibodies. Acta Neuropathol. 91, 647-653.

Massoulié J., and Bon S. (1982) The molecular forms of cholinesterase and acetylcholinesterase in vertebrates. Ann. Rev. Neurosci. 5, 57-106.

Massoulié J., Pezzementi L., Bon S., Krejci E., and Vallette F. M. (1993) Molecular and cellular biology of cholinesterases. Prog. Neurobiol. 41, 31-91.

Master C. L., Simms G., Weinman N. A., Multhaup G., McDonald B. L., and Beyreuther K. (1985) Amyloid plaque core protein in Alzheimer's disease and Down syndrome. Proc. Natl. Acad. Sci. USA 82, 4245-4249.

Méflah K., Bernard S., and Massoulié J. (1984) Interactions with lectins indicate differences in the carbohydrate composition of the membrane-bound enzymes acetylcholinesterase and 5′-nucleotidase in different cell types. Biochimie 66, 59-69.

Mesulam M. M., Geula C., and Morán M. A. (1987) Anatomy of cholinesterase inhibition in Alzheimer's disease, effect of physostigmine and tetrahdroaminoacridine on plaques and tangles. Ann. Neurol. 22, 683-691.

Michaelson S., and Small D. H. (1993) A protease is recovered with a dimeric form of acetyicholinesterase in fetal bovine serum. Brian Res. 611, 75-80.

Mirra S. S., Gearing D. W., McKeel D. W., Crain B. J., Hughes J. P., Vanbelle G., Heyman A., Ball M. J., Clark A. W., Hansen L. A., Hedreen J. C., Joachim C. L., Kim R. C., Kirkpatrick J. B., Markesbery W. R., Davis D., Martinez A. J., Miller C. A., Moossy J.,

Morros J., Nochlin D., Perl D. P., Purohit D., Petito C. K., Rao G. R., et al. (1994) Interlaboratory comparison neuropathology assessments in Alzheimer's disease: A study of the Consortium to Establish a Registry of Alzheimer's Disease (CERAD). J. Neuropath. Exp. Neurol. 53, 303-315.

Moral-Naranjo M. T., Cabezas-Herrera J., and Vidal C. J. (1996) Molecular forms of acetyl- and butyrylcholinesterase in normal and dystrophic mouse brian. J. Neurosci. Res. 43, 224-234.

Morán-Naranjo M. T., Cabezas-Herrera J., and Vidal C. J. (1996) Molecular forms of acetyl- and butyrylcholinesterase in normal and dystrophic mouse brain. J. Neurosci. Res. 43, 224-234.

Morán M. A., Mufson E. J., and Gómez-Romas P. (1993) Colocalization of cholinesterases with b amyloid protein in aged and Alzheimer's brains. Acta Neuropathol. 85, 362-369.

Motter R., Vigopelfrey C., Kholodenko D., Barbour R., Johnsonwood K., Galasko D., Chang L., Miller B., Clark C., Green R., Olson D., Southwick P., Wolfert R., Munroe B., Lieberburg I., Seubert P., and Schenk D. (1995) Reduction of b-amyloid peptide₄₂ in the cerebrospinal fluid of patients with Alzheimer's disease. Ann. Neurol. 38, 643-648.

Navaratnam D. S., Priddle J. D., McDonald B., Esiri M. M., Robinson J. R., and Smith A D. (1991) Anomalous molecular form of acetylcholinesterase in cerebrospinal fluid in histologically diagnosed Alzheimer's disease. Lancet 337, 447-450.

Pfeffer R. I., Afifi A. A., and Chance J. M. (1987) Prevalence of Alzheimer's disease in a retirement community. Am. J. Epidemiol. 125, 420-436.

Probst A., Langui D., and Ulrich J. (1991) Alzheimer's disease: a description of the structural lesions. Brian Pathol. 1, 229-239.

Sáez-Valero J., Tornel P. L., Muñoz-Delgado E., and Vidal C. J. (1993) Amphiphilic and hydrophilic forms of acetyl-and butyrylcholinesterase in human brian. J. Neurosci. Res. 35, 678-689.

Sáez-Valero J., Sberna G., McLean C., Master C. L., and Small D. H. (1997) Glycosylation of acetylcholinesterase as diagnostic marker for Alzheimer's disease. Lancet 350, 929.

Saxena A., Raeh L., Ashani Y., and Doctor B. P. (1997) Structure of glycan moieties responsible for the extended circulatory life time of fetal bovine serum acetylcholinesterase and equine serum butyrylcholinesterase. Biochemistry36, 7481-7489.

Saxena A., Ashani Y., Raveh L., Stevenson D., Patel T., and Doctor B. P. (1998) Role of oligosaccharides in the pharmacokinetics of tissue-derived and genetically engineered cholinesterases. Mol. Pharmacol. 53, 112-122.

Sberna G., Sáez-Valero J., Beyreuther K., Masters C. L., and Small D. H. (1997) The amyloid b-protein of Alzheimer's disease increases acetylcholinesterase expression by increasing intracellular calcium in embryonal carcinoma P19 cells. J. Neurochem. 69,1177-1184.

Sberna G., Sáez-Valero J., Li Q. X., Czech C., Beyreuther K., Masters C. L., McLean, C. A., and Small D. H. (1998) Acetylcholinesterase is increased in the brains of transgenic mice expressing the C-terminal fragment (CT100) of the b-amyloid protein precursor of Alzheimer's disease. J. Neurochem. 71, 723-731.

Schegg K. M., Harrington L. S., Nielsen S., Zwieg R. M., and Peacock J. H. (1992) Soluble and membrane-bound forms of brian acetylcholinesterase in Alzheimer's disease. Neurobiol. Aging 13, 697-704.

Schoenberg B. S., Kokmen E., and Okazaki H. (1987) Alzheimer's disease and other dementing illnesses in a defined United States population: incidence rates and clinical features. Ann. Neurol. 22, 724-729. Shen Z. -X., and Zhang Z. (1993) Anomalous acetyicholinesterase in CSF without clinical diagnosis of Alzheimer's disease. Lancet 342, 62.

Shen Z. X. (1997) An CSF anomolous molecular form of acetylcholinesterase in demented and non-demented subject. Neuroreport 8, 3229-3232.

Siek G. C., Katz L. S., Fishman E. B., Korosi T. S., and Marquis J. K. (1990) Molecular forms of acetylcholinesterase in subcortical areas of normal and Alzheimer disease brain. Biol Psychiatry 27, 573-580.

Silman I., Lyles J. M., and Barnard E. A. (1978) Intrinsic forms of acetylcholinesterase in skeletal muscle. FEBS Letters 94, 166-170.

Small D. H., Michaelson S., and Sberna G. (1996) Non-classical actions of cholinesterases: role in cellular differentiation, tumorigenesis and Alzheimer's disease. Neurochem. Intl. 28, 453-483.

Smith P. K., Krohn R. I., Hermanson G. T., Mallia A. K., Gartner F. H., Provenzano M. D., Fujimoto E. K., Goeke N. M., Olson B. J., and Klenk D. C. (1985) Measurement of protein using bicinchoninic acid. Anal. Biochem. 150, 76-85.

Smith A. D., Jobst K. A., Navaratnam D. S., Shen Z. X., Priddle J. D., McDonald B., King E., and Esiri M. M. (1991) Anomalous acetylcholinesterase in lumbar CSF in Alzheimer's disease. Lancet 338, 1538.

Soreq H., Ben-Aziz R., Prody C. A., Seidman S., Gnatt A., Neville L., Lieman-Hurwitz J., Lev-Lehman E., Ginzberg D., Lapidot-Lifson Y., and Zakut H. (1990) Molecular cloning and construction of the coding region for human acetylcholinesterase reveals a G+C-rich attenuating structure. Proc. Natl. Acad. Sci. USA 87, 9688-9692.

Treskatis S., Christoph E., and Layer P. G. (1992) Butyrylcholinesterase from chicken brain is smaller than that form serum: its purification, glycosylatin and membrane association. J. Neurochem. 58, 2236-2247.

Ulrich J., Meier-Ruge W., Probst A., Meier E., and Ipsen S. (1990) Senile plaques, staining for acetylcholinesterase and A4 protein, a comparative study in the hippocampus and entorhinal cortex. Acta Neuropathol 80, 624-628. Vidal C. J. (1996) Glycosylation of cholinesterases and its alteration in some pathological states. Recent Res. Devell, Neurochem, 1, 37-54.

Wright C. I., Geula C., and Mesulam M. M. (1993) Neuroglial cholinesterases in the normal brain and in Alzheimer's disease, relationship to plaques, tangles and patterns of selective vulnerability. Ann. Neurol. 34, 373-384.

Younkin S. G., Goodridge B., Katz J., Lockett G., Nafziger D., Usiak M. F., and Younkin L. H. (1986) Molecular forms of acetylcholinesterase in Alzheimer's disease. Fed. Proc. 45, 2982-2988.

What is claimed is:

1. A method for the diagnosis of Alzheimer's disease in a patient, comprising the steps of:
(1) providing a sample of an appropriate body fluid from said patient, and
(2) detecting the presence of butyrylcholinesterase with an altered glycosylation pattern in said sample.

2. The method of claim 1 wherein the relative proportions of butyrylcholinesterase with a specific glycosylation pattern to the total butyrylcholinesterase are measured.

3. The method of claim 2 wherein the relative proportions of butyrylcholinesterase are measured using a lectin-binding analysis.

4. The method of claim 3 wherein the lectin-binding analysis includes measurement of butyrylcholinesterase binding to Concanavalin A.

5. The method of claim 4 wherein activity of unbound butyrylcholinesterase is determined.

6. The method of claim 5 further comprising the steps of:
(1) measuring the proportion of acetylcholinesterase binding to Concanavalin A,
(2) measuring the proportion of acetylcholinesterase binding to wheat germ agglutinin,
(3) determining the ratio of acetylcholinesterase unbound to Concanavalin A to acetylcholinesterase unbound to wheat germ agglutinin, and
(4) comparing the ratio with the relative proportion of butyrylcholinesterase unbound to Concanavalin A.

7. The method of claim 6 wherein said ratio is above about 0.95 in Alzheimer's disease patients.

8. The method of claim 6 wherein the total butyrylcholinesterase activity is determined.

9. The method of claim 8 wherein the proportion of butyrylcholinesterase unbound to Concanavalin A is plotted against the total butyrylcholinesterase activity, and the ratio of acetylcholinesterase unbound to Concanavalin A to acetylcholinesterase unbound to wheat germ agglutinin.

10. The method of claim 1 wherein a monoclonal antibody is used to detect the presence of butyrylcholinesterase with an altered glycosylation pattern.

11. The method of claim 1 wherein an abnormal isoform of butyrylcholinesterase with an altered glycosylation pattern is detected.

12. The method of claim 1 wherein said sample is cerebrospinal fluid, blood or blood plasma.

13. The method of claim 12 wherein said blood or blood plasma is prepared from the blood for analysis.

14. The method of claim 13 wherein said body fluid is blood plasma and butyrylcholinesterase is removed prior to analysis for the presence of acetylcholinesterase with an altered glycosylation pattern.

15. An abnormal isoform of butyrylcholinesterase with an altered glycosylation pattern and a lesser affinity for Concanavalin A and a greater affinity for wheat germ agglutinin than butyrylcholinesterase with an unaltered glycosylation pattern.

16. An abnormal isoform of butyrylcholinesterase with an altered glycosylation pattern and a lesser affinity for Concanavalin A and a greater affinity for wheat germ agglutinin than butyrylcholinesterase with an altered glycosylation pattern.

17. The method of claim 8 wherein the ratio of butyrylcholinesterase unbound to Concanavalin A relative to the total butyrylcholinesterase is at least about eight percent.

18. The method of claim 15 wherein said body fluid is blood plasma and butyrylcholinesterase is inactivated prior to analysis for the presence of acetylcholinesterase with an altered glycosylation pattern.
